(19) United States
(12) Patent Application Publication
(10) Pub. No.: US 2005/0085877 A1
(43) Pub. Date: Apr. 21, 2005

(54) Device and process for a controlled irradiation of the human body

(76) Inventor: Walter Kratz, Eitorf (DE)

Correspondence Address: HODGSON RUSS LLP, ONE M & T PLAZA, SUITE 2000, BUFFALO, NY, 14203-2391, US

(21) Appl. No.: 10/922,180

(22) Filed: Aug. 19, 2004

(30) Foreign Application Priority Data
Aug. 19, 2003 (DE) 103 38 004.3

(51) Int. Cl. A61N 5/06

(52) U.S. Cl. 607/94

(57) ABSTRACT

The invention relates to a device (200) for a measurement of the status, as far as relevant for determining the UV irradiation dose, of the skin of a person on his/her skin (cutis), comprising (a) a means (201) for emitting radiation having a defined spectral composition onto the skin (cutis); (b) optionally a means (211) for advancing the radiation of a defined composition to the emission means (201); (a) a sensor (202) for measuring the portion of the radiation irradiated by the emission means (201) which is reflected or absorbed by the skin (cutis); and (d) means (212) for providing energy to the means (201) and/or to the sensor (202) and for transmitting measured data to a data analyzing means (203). The invention also relates to an apparatus (100) and a process for irradiating the body of a user for cosmetic and medical purposes.

### BACKGROUND OF THE INVENTION

The present invention relates to a device and to a process for a controlled irradiation of the human body. In particular, the invention relates to a device and to a process for irradiating the human body with tanning radiation, more particularly with radiation containing a certain portion of ultraviolate (UV) radiation. The device and the process take into account that different persons have different types of skin and experience different sensitivities of their skin against tanning radiation and may be used to being exposed to tanning radiation, in particular to radiation having a certain portion of UV radiation, to different degrees.

Tanning apparatus on the basis of UV radiators are known in several embodiments and sizes. Particularly popular are tanning apparatus allowing a tanning of the whole body. Such Tanning apparatus comprise a lower portion comprising a bed (“bench”) optionally provided with a profile and having installed a number of UV radiator tubes below a surface transparent for tanning radiation, which tubes emit UV radiation. The lower portion of the tanning apparatus corresponds to a movable upper portion which is either pivotable around an axis being located parallel to one of the edges of the bed, usually parallel to one of its long edges, or is movable in a vertical direction on a stand or on a suspension fixed to the ceiling of a room. The upper portion has also installed a number of UV radiator tubes—above a lower surface transparent for tanning radiation—which tubes emit tanning radiation. Users of the tanning apparatus lift or swivel the upper portion thereof into an open position allowing a convenient getting onto the bed, lay down on the bed, close the upper portion and then switch on the UV radiator tubes for a preselected tanning time which may be selected in a time range of a few minutes, but may also be a time of 15 minutes or even more. A tanning process by making use of the above-mentioned apparatus described only with respect to important general features is also known and, hence, is state of the art.

Such tanning devices are used to an increasing extent both in the cosmetic field (both as a private tanning application at home and in so-called “tanning studios” providing professionally used tanning devices for tanning the skin) and in the medical field, where a preventive or therapeutic treatment of the skin is more and more used successfully as a drug-free treatment route or as a treatment route accompanying a drug-involving treatment of the skin (for example in cases of acne, psoriasis etc.).

Basically, tanning apparatus used according to the prior art for all purposes described above are designed in such a manner that a tanning process may be performed without the human skin being damaged by components of the radiation emitted by the radiator tubes, in particular by UV radiation components, provided that habits and protection measures are maintained which a person undergoing a tanning process has to observe also in cases when exposing himself/herself to a natural sun radiation. In other words: In the same way as when tanning in nature, a person having a “white” skin and/or having had few previous opportunities of being exposed to sun radiation and/or artificial UV radiation (e.g. UV radiation of a tanning apparatus) has to select lower radiation doses and/or shorter irradiation times and/or has to protect the skin more with usual (e.g. cosmetic) sun-screens than a person having a “darker” skin and/or having had more previous opportunities of being exposed to the radiation of the sun and/or to artificial UV radiation.

In the medical field, the irradiation dose and/or the irradiation time may be controlled empirically, however in any case under the supervision of a person adjusting the irradiation dose and/or time (e.g. under the supervision of a medical practitioner or of a person instructed by him/her). Usually, for a medical irradiation treatment, a low constant irradiation dose and/or a short treatment time is selected at the beginning of an irradiation treatment, and the skin reaction of the irradiated person to the radiation is detected. In accordance with such a primary reaction, the dose and the time, respectively, are adjusted for the subsequent steps of the irradiation treatment and, usually, are raised slowly. Such an adjustment requires a certain experience, i.e. experience with the method of treatment in general and with the evaluation of the physiological reaction achieved and with the operation of the tanning apparatus used. Different reactions of the skin to an irradiation and different types of skin can only be met by a reduction of the (usually constant) irradiation dose or a shortening of the irradiation time.

In the cosmetic field, in particular when tanning apparatus are provided on a professional basis, usually persons are available to advise the customer with respect to the tanning time and the irradiation dose. Often, however, such advice is not accepted. In the private area, such advice is not available at all. Quite to the contrary: The person intending to use the tanning apparatus selects, on his/her own volition, the tanning time and irradiation dose of the apparatus in most of the cases. Hence, even in cases where skilled advice was given before, this does not reliably prevent a stronger irradiation dose or a longer tanning time, respectively, from being selected, for example in erroneously desiring to accelerate the process of tanning or to achieve a darker tan within one treatment “session”. As in known from nature, this results into erythemias caused on the skin by the UV radiation, which are known under the term “sunburn” and which may be very painful and may damage the skin in the long run.

The document DE-A43 12 547 describes tanning devices allowing the risks of a too intensive irradiation of the skin to be diminished substantially both in the medical field and in the cosmetic field. In accordance with the teaching of the document, this may be accomplished by means of an adaptation of the UV radiation of a tanning device to the individual type of skin of the respective user. The UV radiator tubes are provided with separate means for controlling their power, and several of those power control means are connected to a central control means allowing an individual control of single radiator tubes. Practically, the power control is achieved by a means for determining the individual skin color of the person to be subjected to irradiation (which means has per se been known for a long time). Immediately on the skin of the person to be subjected to the irradiation, the type of skin and the skin color, respectively, of the person to be irradiated are detected optically by means of a scanning head. This is performed by irradiating light of a well-defined spectral composition, scanning of the light reflected by the skin and determining the ratio of the light absorbed/reflected to the light irradiated. The data obtained (and compared to empirical data) are fed to the central processing unit, and the maximum irradiation dose which the person to be irradiated may receive is determined, as is also determined—depending upon the desired duration of the irradiation—the maximum irradiation time applicable in each individual case.

It is a disadvantage of the above proposal that the fact is not taken into account that different parts of the human body are susceptible to tanning radiation to a different degree and also have a different sensitivity to tanning radiation, in particular UV radiation. For example, the body (and in particular, those parts of the body which usually are covered by clothes) are considerably more sensitive to UV radiation than the face. A measurement of the skin tan or the skin type, respectively, at the face regularly result into a higher maximum dose of allowed radiation by means of the device described in the document DE-A 43 12 547 than a measurement of the skin tan or skin type, respectively at the body, for example at the chest or at the belly. The consequences of such “mismeasurement,” although correct for the skin type measured at the respective part of the skin, are too high or too low radiation doses at other parts of the skin of the same person. Specifically for the above-selected case of a primary measurement of the skin type or the skin tan, respectively, at one of the regularly less sensitive parts of the skin (e.g. at the face of the person to be subjected to irradiation), the body is (and more sensitive parts of the body particularly are) subjected to a too strong dose of tanning radiation, which fact results into the known erythema irritation of the skin or event into a damage of the sensitive skin.

### SUMMARY OF THE INVENTION

The invention contributes to abolishing this disadvantage of the prior art. Surprisingly, it was found that it is possible to avoid an over-exposure and to make optimal use of the maximum irradiation dose admissible for certain parts of the skin by conducting several measurements of the skin type and of the status of the skin tan, respectively, of a person to be tanned at different parts of the skin and by a separate adjustment of the radiation impinging on the respective part of the body in accordance with the results of the respective measurements by a separate control of the UV radiation sources emitting the radiation, e.g. of the UV radiator tubes.

Hence, the invention relates to a device for a measurement of the status as far as relevant for determining the UV irradiation dose, of the skin of a person on his/her skin (cutis) comprising (a) a means for emitting radiation having a defined spectral composition onto the skin (cutis); (b) optionally a means for advancing the radiation of a defined composition to the emission means; (c) a sensor for measuring the portion of the radiation irradiated by the emission means which is reflected or absorbed by the skin (cutis} as well as (d) means for providing energy to the means and/or the sensor and for transmitting measured data to a data analysing means. In a particularly preferred embodiment of the invention, the measurement of the status, as far as relevant for determining the UV irradiation dose, of a person's skin comprises the measurement of the skin color of said person and/or the measurement of the skin tan status of said person.

Furthermore, the invention relates to an apparatus for irradiating radiation comprising a UV radiation portion onto the body of a user, said apparatus comprising at least one means having a surface facing the user and being transparent for the radiation and having several groups of UV radiation sources arranged on that side of the surface which is turned away from the user, which UV radiation sources are designed with respect to their number and position, in such a way that they can direct radiation comprising a UV radiation portion substantially separately onto different parts of the user's body, and are controllable by one or more means for controlling the power of the UV radiation sources, wherein the power control is effected in accordance with the results of the measurements of the status, as far as relevant for determining the UV irradiation dose, of the user's skin at several points of his/her cutis.

Furthermore, the invention relates to a process for irradiating to a user's body radiation comprising a UV radiation portion from UV radiation sources emitting such radiation, said process comprising the step of measuring the status, as far as relevant for determining the UV irradiation dose, of the user's skin at several points of the cutis, and controlling the power of single groups of said UV radiation sources, which can direct their radiation substantially separately onto different parts of the user's body, in accordance with the results of said measurements.

Furthermore, the invention also relates to such irradiation processes for cosmetic and/or medical purposes.

Finally, the invention also relates to the use of the above device for measuring the status, as far as relevant for determining the UV irradiation dose, of a person's skin in connection with the irradiation, onto the skin, of radiation comprising a UV radiation portion in a tanning apparatus having artificial UV radiation sources and/or in nature, and/or in connection with the medical irradiation, onto the skin, of radiation comprising a UV radiation portion and/or in connection with the cosmetic irradiation, onto the skin, of radiation comprising a UV radiation portion.

The invention is further explained below by means of the preferred embodiments shown in the figures. The embodiments shown in the figures serve as an explanation of the invention and not as a restriction of the same.

### BRIEF DESCRIPTION OF THE DRAWING FIGURES

FIG. 1 shows a device for measuring the status, as far as relevant for determining the UV irradiation dose, of a person's skin, particularly for measuring the skin color and/or the skin tan status of a person in a perspective view;

FIG. 2 shows a device according to FIG. 1 in an exploded view;

FIG. 3 shows a device according to FIG. 1 in a front view (FIG. 3A), in a side view (FIG. 3B) and in a plan view (FIG. 3C) as well as in a side sectional view (FIG. 3D) and in a longitudinal sectional view (FIG. 3E);

FIG. 4 is a side sectional view of the functional area of the device according to FIG. 1;

FIG. 5 shows an apparatus for irradiating radiation comprising a UV radiation portion onto the human body, specifically considering the device for measuring the skin color and/or the skin tan status;

FIG. 6 shows a circuit diagram representing the steps of addressing UV radiation sources and, in particular, UV radiation tubes by analysing the results of the skin color measuring device; and

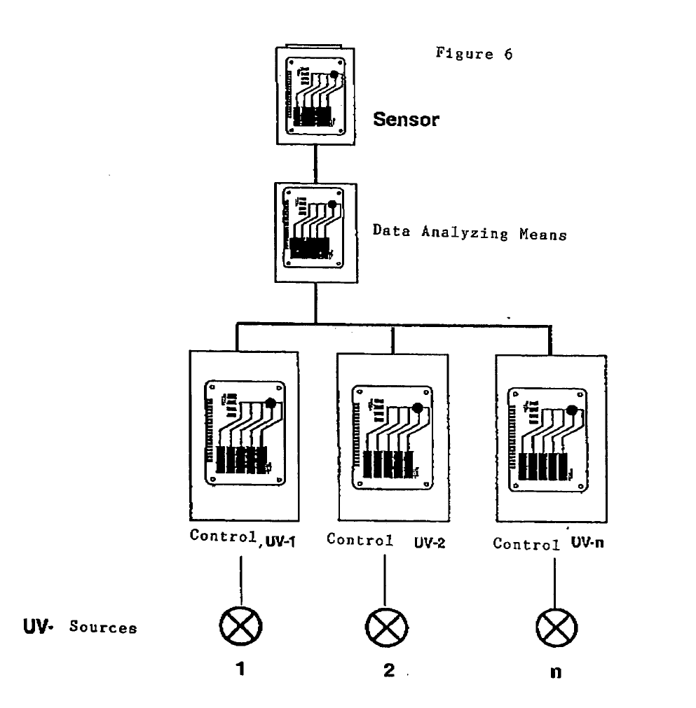

FIG. 7 shows a flow diagram showing the course of the process for irradiating the human body with a previous and/or simultaneous determination of the skin color and/or the skin tan status.

### DETAILED DESCRIPTION OF THE INVENTION

The invention is now further described by referring to the figures in detail.

The tem “skin color” is understood, in the context of the present invention, as the sum of all wavelengths of reflected light which is reflected from the skin surface or cutis as a consequence of an irradiation of light having a defined intensity and wavelength. The skin color serves as a basis for evaluating the question as to which ultraviolet radiation dose can be tolerated by the skin without it being irritated by an erythema, not even in its first signs. It was found that the skin of different persons reacts in different ways to light having a defined spectral composition and being irradiated onto the skin for a measurement of the skin status, as far as relevant for determining the UV irradiation dose. The skin of persons having a white skin color usually reacts in a clearly more sensitive way (and, hence, tolerates clearly less UV radiation before reaching the erythema irritation threshold) than the skin of people having a dark skin color. The light reflected by the surface (cutis) of the skin having a specific skin color provides a good measure for the skin sensitivity towards UV radiation as it is used for the process of tanning the skin.

In contrast to the term “skin color” the term “skin tan status” is understood to mean in the context of the present invention, the status of the skin at the time of measurement, as caused by a pigmentation which is achieved as a reaction to the irradiation of the skin by UV radiation. It is known from previous experience that the pigmented (dark) skin is less sensitive to UV radiation than less pigmented (white) or even unpigmented skin. Independent from the skin color, the skin tan status provides another good measure for the UV radiation dose which the skin of a person may receive without being irritated by erythemas.

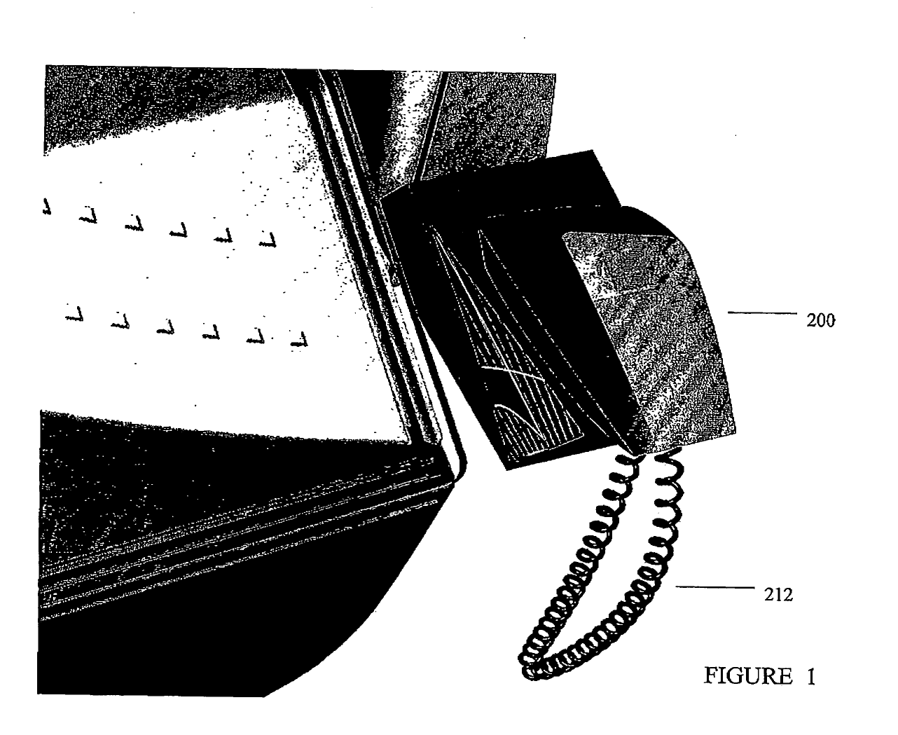

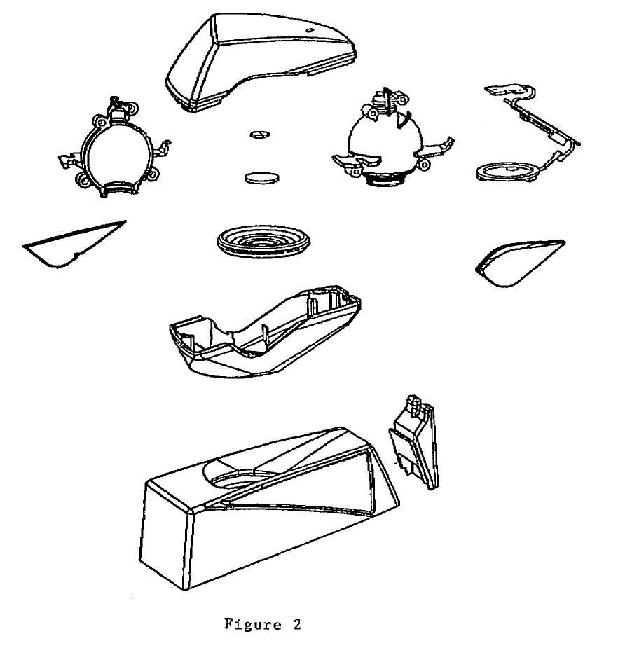

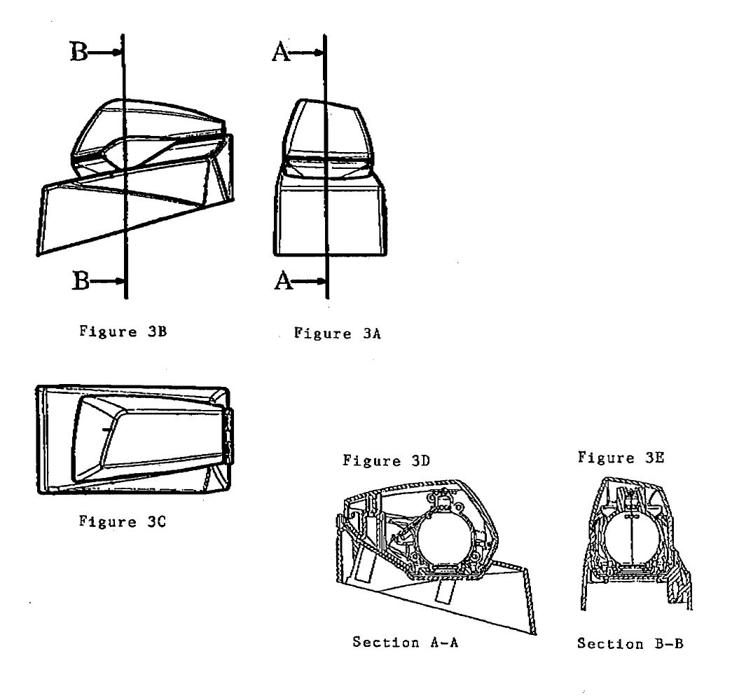

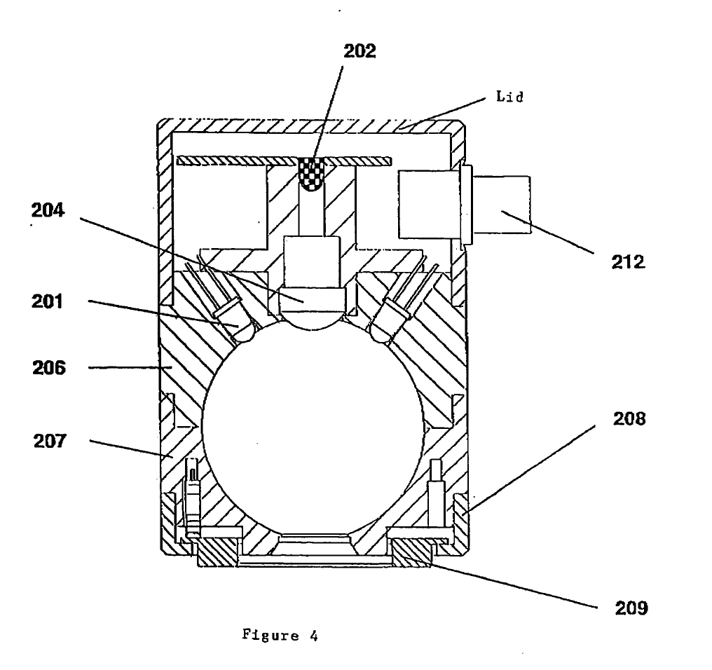

The device 200 according to the invention for a measurement of the status, as far as relevant for determining the UV irradiation dose, of the skin of a person and—particularly preferred—for a measurement of the skin color and/or of the skin tan status of a person, is shown in FIGS. 1 to 4. In general, the skin color of a person and/or the skin tan status of a person is/are measured by means of a device 200 comprising: (a) a means 1001 for emitting radiation having a defined spectral composition onto the skin (cutis); (b) optionally a means 211 for advancing the radiation of a defined composition to the emission means 201; (c) a sensor 202 for measuring the portion of the radiation irradiated by the emission means 201 which is reflected or absorbed by the skin (cutis) as well as (d) a means 212 for providing energy to the means 201 and/or to the sensor 202 and for transmitting measured data to a data analysing means 203.

In preferred embodiments of the invention, the means 201 for releasing or emitting, respectively, radiation having a defined composition may be one or more light emitting diodes (LEDs) or a light guide. The former may be used as a single LED or in groups of LEDs; the latter may (but not necessarily has to) be different with respect to the wavelength of the emitted light. A light guide advances light of a defined spectral composition from a light source (which usually is spaced away from the emitting end of the guide). In accordance with the invention, the use of several LEDs is particularly preferred which are different from each other with respect to the wavelength of the emitted light. In a particularly preferred embodiment according to the invention, three LEDs are used. The three LEDs may emit, for example, blue, green and red light (which is, however, not compulsory and, hence, not restrictive).

The term “radiation having a defined spectral composition” as used in the specification and claims, is understood to mean, in the context of the present invention, radiation the wavelengths of which are selected from the continuous spectrum of the light, particularly preferred of the visible light, i.e. of the light having wavelengths of 400 to 800 nm in such a way that the irradiated light can be reflected by the skin (cutis) and provides usable results of the measurements of the status of the skin, as far as relevant for determining the UV radiation dose, and, particularly, a measurement of the skin color and of the skin tan status, respectively. In the present invention, it is possible to use radiation having wavelengths extending over the whole spectrum, particularly radiation of the visible light spectrum, or to use monochromatic radiation. In particularly preferred embodiments of the invention, light of specific wavelengths is used, for example light of wavelengths in the range of from about 400 to 450 nm (blue), 470 to 520 nm (green), and 700 to 750 nm (red). Having in mind reproducible results, particularly of the measurements of the skin color and/or the skin tan status, the emission of light in the above-referenced three areas of wavelengths is particularly approved.

The light of a defined spectral composition is provided by LEDs as the emission devices 201 in a further preferred embodiment of the invention. Those LEDs may be diminished in size so far that they may be accommodated as single or plural ones in the device 200 and, despite their small size, reliably provide the light of a defined spectral composition and emit it to the skin.

In an alternative embodiment of the device 200 according to the invention, the light of a defined spectral composition later to be emitted to the skin (cutis) is generated at a place spaced from the device. The light thus generated—possible is a narrow or a broad spectral distribution of the wavelengths—is guided to the emission means 201 by one or more light guide(s) and is emitted to the skin from said device 201.

In a particularly preferred embodiment of the invention, the device 200, in the area of emitted radiation, comprises another device 205 serving the diffusion of the emitted light between the emission device 201 and the skin (cutis) and, hence, changing the radiation direction from the single directed beam to a plurality of diffuse beams. Particularly, a sphere-shaped cavity 205 within the deice 200 has proven advantageous, which cavity serves as a diffusor 205 and changes the direction of the emitted radiation between the emission device 201 and the skin (cutis).

In a further embodiment of the invention, a so-called “Ulbricht sphere” is used as the diffusor 205. This is advantageous in generating a diffuse radiation very similar to the radiation of natural sunlight. For irradiating light for a measurement of the status, as far as relevant for determining the UV irradiation dose, of the skin of a person, this solution has the advantage that a measurement radiation is irradiated which is very similar to that of a natural environment.

In accordance with the invention, the device 200 further comprises a sensor 202 serving a measurement of the radiation reflected by the skin (cutis) or serving the measurement of the irradiation absorbed by the skin (cutis) (in the sense of the equation: irradiated radiation minus reflected radiation is equal to absorbed radiation), based on the radiation irradiated by the emission device 201. As the sensor 202, any sensor or detector may be used which a person skilled in the area of measuring light in accordance with its wavelength and/or intensities knows. Usual photodiodes are preferred as an alternative, photographic cameras (optionally miniaturized) in a form known to a skilled person may be used, for example a linear-array camera or optical camera. In particularly preferred embodiments of the invention, the sensor 202 may be used together with additional optical devices, for example with one or more lens(es) 204 for focussing the radiation reflected and/or with one or more prism(s) for splitting the reflected radiation into radiation distinguished by wavelengths in order to be in the position to detect radiation of one or more defined wavelength(s) in the light reflected from the skin (cutis). The sensor 202 takes a measurement of the radiation reflected from the skin (cutis) dependent from the status relevant for determining the UV irradiation dose for the skin of a person. Particularly, it was found that, depending upon the color of the skin or, respectively, upon a pigmentation already occurred by a previous irradiation of tanning UV radiation, different amounts of irradiated radiation having a defined spectral composition are reflected by the skin. More in detail: white or not yet pre-pigmented or moderately pre-pigmented skin reflects more radiation than dark skin or skin already pre-pigmented. The dependency of the amount of reflected radiation, based on a defined amount of irradiated radiation having a defined spectral composition, allows an exact assessment of the irradiation dose and/or the irradiation time with a surprisingly high reliability. Particularly, the invention takes into account that different-parts of a person's body have a different status of the skin, as far as relevant for determining the UV irradiation dose, specifically have a different skin color, and/or have a different skin tan status. This may, for example, result from the fact that every day a specific part of the body (for example the face or the arms) is/are exposed to tanning UV radiation more often than another part of the body (for example the chest or the back) and, hence, they have another skin color and another skin tan status, respectively. Based on, for example, a pre-pigmentation previously conducted, the face and the arms may receive stronger UV radiation doses and/or may be irradiated for a longer time than the chest and the back. A measurement of the status, as far as relevant for determining the UV irradiation dose, of the respective parts of the skin, for example, preferably a measurement of the skin color or the skin tan status, respectively, by means of an emission of light having a defined spectral composition and the subsequent measurement of the amount of reflected light (reflected, on the one hand, by the pre-tanned and, hence, darker parts of the skin, of the face and of the arms and, on the other hand, by those parts of the skin not having received a tanning and, hence, being lighter, as for example, the skin of the chest or of the back) provides a surprisingly reliable standard for evaluating the UV irradiation dose which may be irradiated onto the skin or for a possible time of irradiating UV radiation having a defined intensity without exceeding the erythema threshold.

The amount of reflected radiation determined by the sensor 202 is transmitted via means 212 comprised by the device 200 to a data analysing means 203. It is the purpose of this means to provide, in addition to an analysis of the data received, information about the intensity and duration of a possible irradiation of certain parts of the body with UV radiation and to thereby initiate a control for exposing certain parts of the body to less intensive UV radiation and to expose other parts of the body to stronger tanning UV radiation. In accordance with the invention, the means 212 may also provide the necessary energy to the sensor.

In a preferred embodiment of the invention, the device 200 additionally comprises a means 209 for adjusting defined distances between the emission means 201 and the skin (cutis) on the one hand, and the sensor means 202 and the skin (cutis), on the other hand. In an advantageous way, this is practically realised by connecting the corresponding means in a fixed arrangement, for example in a measuring head or measuring sensor. By such a means, a specific arrangement of the respective means for improving the precision of the measurement and for facilitating the practical operation of the device 200 during a measurement is achieved.

The device 200 constructed in accordance with the above description and optionally comprising further means as, for example, microswitches, cables, plugs, and/or an unbreakable and humidity-resistant housing may be used for a measurement of the status as far as relevant for determining the UV irradiation dose, of the skin of a person in a number of applications. Such a measurement of the status of the skin is conducted, for example, in connection with an irradiation of the skin with radiation comprising a UV radiation portion in a usual tanning apparatus having artificial UV radiation sources as, for example, UV radiator tubes or high pressure burners. The intensity of those UV radiation sources may, in particularly preferred embodiments of the invention, be controlled in accordance with the status, as far as relevant for determining the UV irradiation dose, of the skin of the user in such a tanning apparatus. In a particularly preferred embodiment, such control may be conducted separately for different parts of the body in accordance with their skin color or their skin tan status, respectively. Thus, darker and/or pre-pigmented parts of the skin, e.g. the skin of the face and/or of the arms, may be irradiated with a stronger intensity and/or for a longer period of time than whiter and/or less (or not at all) pre-pigmented parts of the skin (chest, back). The invention is described in relation to the latter embodiment, without being restricted to this embodiment.

Another area of application for the device 200, which is also relevant in accordance with the invention and is, hence, a preferred area of application, is the measurement of the status, as far as relevant for determining the UV irradiation dose, of the skin of a person in connection with an irradiation of radiation comprising a UV radiation portion in the nature. Specific examples for this embodiment (without the invention being restricted to those embodiments) may be all preparatory steps for a stay on the beach or on the water (sailing, surfing) or for sports activities where a natural sun ray irradiation having a natural UV portion has to be expected.

A further application of the device 200, which is also preferred in accordance with the invention, is a measurement of the status, as far as relevant for determining the UV irradiation dose, of the skin of a person in connection with a medical irradiation of the skin with radiation comprising a UV radiation portion. Certain diseases of the skin as, for example, acne or psoriasis (to name only two of them) may be cured by irradiating UV radiation to the skin struck by said disease or at least, may be moderated considerably with respect to their symptoms. Often, a UV irradiation accompanies the administration of medicaments or may allow the drug dose administered to be reduced or may even replace such drug administration. A controlled irradiation, i.e. an irradiation adjusted, with respect to intensity and duration, to specific parts of the body struck by said disease, may accelerate the curing process, particularly at those parts of the body struck by the disease, without the erythema risk being increased, which risk prevents a continuous healing.

Another area of application for the device 200, which is also preferred in accordance with the invention, is a measurement of the status, as far as relevant for determining the UV irradiation dose, of the skin of a person in connection with a cosmetic irradiation of the skin with radiation comprising a UV radiation portion. A tanning of the skin for purely cosmetic reasons, for improving the appearance, for providing a more healthy and firm skin and for strengthening a good health (optionally also for preparing a sojourn in climates different from at home, where a more intensive exposition of the body to irradiation is expected) gain more and more emphasis. In such a case, a protection against excessive UV radiation becomes more important The present invention, in a particularly advantageous way, provides good preconditions for avoiding a too strong irradiation and contributes to avoiding erythemas, which are contrary to the aim of an application of UV irradiation in the cosmetic area.

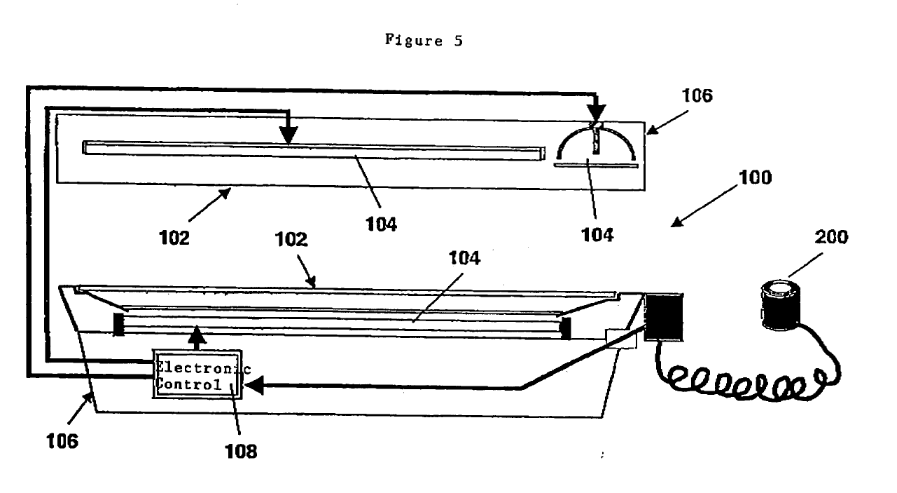

Reference is now made to FIG. 5. In accordance with the present invention, there is also provided an apparatus 100 for irradiating radiation comprising a UV radiation portion onto the body of a user. The apparatus 100 comprises at least one means 106 having a surface 102 facing the user and being transparent for the radiation, said apparatus having several groups of UV radiation sources 104 arranged on that side of the surface 102 which is turned away from the user, which UV radiation sources 104 are designed, with respect to their number and position, in such a way, that they can direct radiation comprising a UV radiation portion substantially separately onto different parts of the users body and which are controllable by one or more means 108 for controlling the power of The UV radiation sources 104, wherein the power control is effected in accordance with the results of the measurements of the status, as far as relevant for determining the UV irradiation dose, of the user's skin at several points of his/her skin (cutis).

In a preferred embodiment of the invention, the apparatus 100 may be a usual tanning apparatus the UV radiation sources 104 of which are designed, in accordance with the aims of the present invention, in such a way that single groups of UV radiation sources 104 may be directed onto different parts of the user's body, and that the respective UV radiation sources 104 directed to different parts of the user's body may be controlled separately with respect to the irradiation intensity and/or radiation duration. As UV radiation sources 104, there may be used, in particularly preferred embodiments of the invention, e.g. UV radiation tubes or high pressure burners.

In accordance with the invention, a control of the power of the UV radiation sources 104 is effected in accordance with the results of measurements of the status, as far as relevant for determining the UV irradiation dose, of the skin of the person using the apparatus 100, e.g. the tanning apparatus 100, before an irradiation and/or simultaneously with the irradiation. In a preferred embodiment of the apparatus 100 according to the invention, a control of the power of the UV radiation sources 104 is effected in accordance with the results of the measurements of the skin color and/or of the skin tan status of the user at the time of interest. In an even more preferred embodiment of the invention, such a measurement of the skin color and/or of the skin tan status of the user is effected by means of one or more devices 200 as they are described above in detail by referring to FIGS. 1 to 4. On the basis of the results of the measurements achieved with the device(s) 200, which determine the absorption and/or reflection of radiation having a defined spectral composition and radiated onto the skin, in relation to the overall radiation irradiated, the devices 108 in the apparatus 100 are addressed, which control the power of the UV radiation sources 104.

In another preferred embodiment of the invention, the number of groups of UV radiation sources 104 in the apparatus 100 is at least equal to or even larger than the number of points of the user's skin (cutis) where a measurement of the status, as far as relevant for determining the UV irradiation dose, of the user's skin is effected, more preferred where measurements of the skin color and/or of the skin tan status of the user's skin are effected. In other words: for a certain apparatus 100, it has to be decided at how many points of the skin (cutis) of the user of the apparatus 100 measurements of, for example, the skin color and/or of the skin tan status are conducted, i.e. how many different steps of tanning of the user's body should be selected on the basis of the condition of the users skin. In accordance with such a decision, the number of UV radiation sources separately controllable with respect to their intensity or radiation duration, respectively, is selected to be equal to or larger than the number of points of measurement in order to achieve a tanning of the skin exactly adapted to the status of different parts of the user's body without taking a risk of erythemas occurring due to a too strong irradiation of UV radiation. To give just a few examples: it is advantageous in accordance with the invention to provide groups of means 108 for controlling the power and/or the irradiation intensity of UV radiation sources 104 in accordance with the results of measurements of the status, as far as relevant for determining the UV irradiation dose, of the user's skin, in particular measurements of the skin color and/or of the skin tan status of the user of the apparatus 100, at several points of his/her skin (cutis), which are selected from skin of the face, skin of the chest, skin of the back, skin of the belly, skin of the bottom, skin of the arms and skin of the legs.

In accordance with another preferred embodiment of the invention, the apparatus 100 may be controlled in such a way that the device(s) 200 take(s) a measurement of the skin color and/or of the skin tan status of the user before starting the irradiation of UV radiation. In accordance with the result of the measurement, the intensity of the UV radiation sources 104 and/or the duration of the irradiation are controlled by means of the device(s) 108 in a manner adjusted to the skin color and/or to the skin tan status of the user.

In another embodiment of the invention which improves the co-ordination and, hence, is further preferred, the device(s) 200 effect(s) a measurement of the skin color and/or the skin tan status of the user not only before beginning the irradiation, but additionally at least once in the course of the irradiation, further preferred intermittently or continuously in the course of the irradiation. This embodiment has the advantage of allowing to achieve results of measurements also during the irradiation which results come up as a reaction to the irradiation, so that changes of the parameters (skin color, skin tan status) resulting from a UV irradiation in the course of the treatment may be taken into account.

In a particularly preferred embodiment, the apparatus 100, which, for example, may be a usual tanning apparatus and may have the features described above in detail, may comprise
(a) a bed 110 with a surface 112 for the user transparent to radiation, said bed having UV radiation sources 114 emitting radiation;
(b) a movable upper part 120 with a protection surface 122 transparent for the radiation and having UV radiation sources 124 emitting such radiation;
(c) wherein the upper part 120, in relation to the bed 110, may be opened for allowing a user to get in and may be closed before starting the irradiation step;
(d) control means 130 allowing a user to control the time of the irradiation step; and
(e) one or more means 108 for allowing a separate control of the power of the UV radiation sources 114, 124, where the control of the power is effected in accordance with the results of measurements of the skin color and/or of the skin tan status of the user at several points of his skin (cutis).

The above-described apparatus 100 is not restricted to the above-referenced embodiment with respect to its design as a usual tanning apparatus wherein a user is lying on a bed and is irradiated by UV radiation emitted from UV radiation sources 114,124, which are positioned below the bed's surface 112 and above the protection surface 122. Quite to the contrary, the above-described embodiment does also comprise so-called stand-up tanning apparatus. In such a stand-up tanning apparatus, the UV radiation sources are arranged vertically, and the user is standing and is surrounded by two “halves” of the apparatus 100 wherein the UV radiation sources 114, 124 are arranged around the user.

In accordance with the invention, there is also claimed a process for irradiating to a user's body, radiation comprising a UV radiation portion from UV radiation sources emitting such radiation. Said process comprises the step of measuring the status, as far as relevant for determining the UV irradiation dose, of the user's skin at several points of the cutis, and, in particular, measuring the skin color and/or the skin tan status of the user's skin, and controlling the power of single groups of said UV radiation sources, which can direct their radiation substantially separately onto different parts of the user's body, in accordance with the results of said measurements.

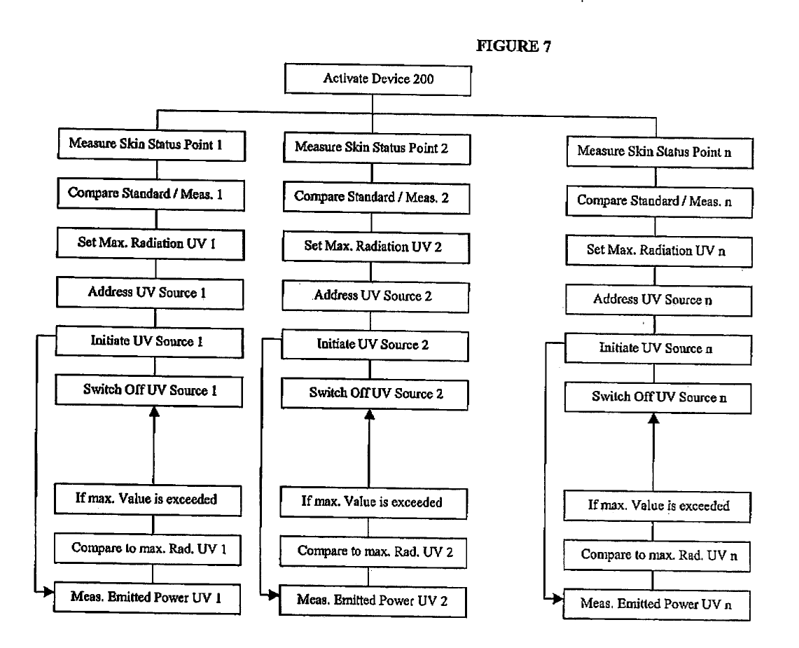

In a specific case of the measurement of the skin status, as far as relevant for a UV irradiation, the device 200—as is shown in the flow scheme in FIG. 7—is activated, and a measurement of the skin status of the user is effected at at least two points of the user's body subsequently, generally at n points of the user's body. Those points may be selected, for example, from skin of the face, skin of the chest, skin of the back, skin of the belly, skin of the bottom, skin of the arms and skin of the legs without restricting the invention to such measurements. The amount of radiation reflected by the user's skin is measured by the sensor 202, and the data obtained are transmitted to the data analyzing unit 203. In said unit, the values of the measurements of the amount of radiation at the measurement point 1, at the measurement point 2, . . . and at the measurement point n are compared to standard values from which can be concluded how high the maximum radiation dose (or the maximum irradiation time at a certain dose) may be at the point where the first, second, . . . , n-th measurement was effected. The UV irradiation dose for the first second, . . . , n-th point of the user's body is adjusted accordingly, wherein the adjustment may be an adjustment of the intensity at a constant time of the whole irradiation, an adjustment of the time at a constant irradiation dose or any combination of parameters providing a guarantee that the skin is irradiated with such an intensity only that the erythema threshold is not reached. The UV radiation source, for example the UV radiation tube or the high-pressure burner, for the point 1,2, . . . , n is initiated and is switched off again after reaching the maximum value of the dose, of the time and/or of any other parameter desired.

In a particularly preferred embodiment of the invention, the emitted power and/or the emitted UV radiation since the initiation of the radiation emission may also be measured in the course of the irradiation at the points 1, 2 . . . , n of the user's body and may then be compared with the values indicated above to be maximum values. If such a comparison shows that the maximum values for a specific point 1, 2, . . . , n, are reached or even exceeded the tanning apparatus will switch off the UV radiation sources for an irradiation of that point or those parts of the body singly (or may switch off the whole apparatus). This alternative embodiment is shown in the lower third of FIG. 7.

The number of points of the user's skin (cutis) where a measurement of the relevant parameters as, for example and preferrably, the skin color and the skin tan status of the user, may be effected is basically not restricted. It is, however, preferred that the skin color and/or the skin tan status of the user be measured at at least two points of his/her skin (cutis), and the power of two or more groups of UV radiation sources is controlled in accordance with the results of such measurements. It is particularly preferred in the process according to the invention that the skin color and/or the skin tan status of the user be measured at least at two points, generally at n points of his/her skin (cutis), which are selected from skin of the face, skin of the chest, skin of the back, skin of the belly, skin of the bottom, skin of the arms and skin of the legs.

In accordance with the process according to the invention, it is particularly preferred that the measurement of the skin color and/or of the skin tan status of the user be effected by means of one or more means 200 measuring the absorption and/or reflection of radiation having a defined spectral composition irradiated onto the skin in relation to the irradiated radiation and that the means 108 for controlling the power of the UV radiation sources 104 are addressed in accordance with the results of such a measurement, particularly as shown in FIG. 7.

As already pointed out above in connection with the apparatus 100, the measurement of the status, as far as relevant for determining the UV radiation dose, of the skin of a user is effected before starting the irradiation in particularly preferred embodiments of the process according to the invention. In accordance with the results of such measurements, the irradiation is controlled. In another embodiment of the invention, which may also be realized advantageously, a measurement of the parameters, preferably a measurement of the skin color and/or of the skin tan status of the user, is effected before starting the irradiation and, additionally, at least once in the course of the irradiation, even more preferred intermittently or continuously in the course of the irradiation. This embodiment is particularly advantageous, since, in the course of the irradiation of radiation containing a UV portion it may be prevented, in emergency cases, by switching off the corresponding radiation sources or even the whole apparatus, that the maximum parameters of the irradiation (time, intensity, etc.) possibly resulting into the generation of erythemas are exceeded, if a measurement of the parameters relevant for the skin shows that the skin at the measurement point is not suitable for such a long and/or intensive irradiation of UV radiation.

The process of the invention is applied in numerous fields which are not restricted in accordance with the invention. Preferred application fields are processes for a cosmetic skin irradiation and for a medical skin irradiation.

By a measurement of the status, as far as relevant for determining the UV radiation dose, of the user's skin, preferably by a measurement of the skin color and/or a skin tan status of the user, at least before starting the irradiation, it is possible to adjust an irradiation of ultra-violet radiation exactly to the individual parameters of parts of the user's skin having a different sensitivity to UV radiation. An over-exposure resulting into the generation of an erythema (“sunburn”) is reliably prevented even for sensitive light or unpigmented skin. This is achieved by a measurement process using the device 200 of the present invention which is simple, quickly to effect and does not need extensive apparatus.

1. A device (200) for a measurement of the status, as far as relevant for determining the UV irradiation dose, of the skin of a person on his/her skin (cutis), comprising (a) a means (201) for emitting radiation having a defined spectral composition onto the skin (cutis); (b) optionally a means (211) for advancing the radiation of a defined composition to the emission means (201); (c) a sensor (202) for measuring the portion of the radiation irradiated by the emission means (201) which is reflected or absorbed by the skin (cutis); and (d) means (212) for providing energy to the means (201) and/or to the sensor (202) and for transmitting measured data to a data analyzing means (203).

2. The device (200) according to claim 1, further comprising a means (209) for adjusting defined distances between the emission means (201), the sensor means (202) and skin (cutis).

3. The device (200) according to claim 1 or claim 2, further comprising a means (205) for diffusing the emitted radiation between the emission means (201) and the skin (cutis).

4. The device (200) according to claim 3, comprising a sphere-shaped cavity in the inner part of the device or an Ulbricht sphere as the diffusion means 205.

5. The device (200) according to any of the claims 1 to 4, wherein the measurement of the status, as far as relevant for determining the UV irradiation dose, of a person's skin comprises a measurement of the skin color of said person and/or a measurement of the skin tan status of said person.

6. An apparatus (100) for irradiating radiation comprising a UV radiation portion onto the body of a user, said apparatus comprising at least one means (106) having a surface (102) facing the user and being transparent for the radiation and having several groups of UV radiation sources (104) arranged on that side of the surface (102) which is turned away from the user, which UV radiation sources (104) are designed, with respect to their number and position, in such a way that they can direct radiation comprising a UV radiation portion substantially separately onto different parts of the user's body, and are controllable by one or more means (108) for controlling the power of the UV radiation sources (104), wherein the power control is effected in accordance with the results the measurements of the status, as far as relevant for determining the UV irradiation dose, of the user's skin at several points of his/her cutis.

7. The apparatus (100) according to claim 6, wherein the power control is effected in accordance with the measurements of the skin color and/or of the skin tan status.

8. The apparatus (100) according to claim 6 or claim 7, wherein the number of groups of UV radiation sources (104) is larger than or equal to the number of points of the user's cutis where measurements of the status, as far as relevant for determining the radiation dose, of the user's skin are effected, preferably where measurements of the user's skin color or the user's skin tan status are effected.

9. The apparatus (100) according to any of the claims 6 to 8, comprising several means (108) for controlling the power of the UV radiation sources (104) in accordance with the results of the measurements of the user's skin type and/or the user's skin tan status at several points of the skin (cutis) selected from skin of the face, skin of the chest, skin of the back, skin of the belly, skin of the bottom, skin of the arms and skin of the legs.

10. The apparatus (100) according to any of the claims 6 to 9, wherein the measurement of the skin color and/or of the skin tan status of the user is effected by means of one or more means (200) measuring the absorption and/or reflection of radiation having a defined spectral composition irradiated onto the skin in relation to the irradiated radiation and the means (108) controlling the power of the UV radiation sources (104) are addressed in accordance with results of said measurement.

11. The apparatus (100) according to any of the claims 6 to 10, wherein the means (200) take(s) a measurement of the skin color and/or of the skin tan status of the user before beginning the irradiation.

12. The apparatus (100) according to claim 11, wherein the means (200) take(s) additional measurements of the skin color and/or of the skin tan status of the user additionally at least once in the course of the irradiation, preferably intermittently or continuously in the course of the irradiation.

13. The apparatus (100) according to any of the claims 6 to 12, comprising
(a) a bed (110) with a surface (112) for the user transparent for the radiation, and UV radiation sources (114) emitting radiation;
(b) a movable upper part (120) having a protection surface (122) transparent for the radiation, and UV radiation sources (124) emitting radiation;
(c) wherein the upper part (120), in relation to the bed (110) may be opened for allowing a user to get in and may be closed before starting the irradiation process;
(d) controlling means (130) for controlling the time of an irradiation process which may be used by the user; and
(e) one or more device(s) (108) for separately controlling the power of the UV radiation sources (114, 124), wherein the control of the power is effected in accordance with the results of measurements of the skin color and/or of the skin tan status of the user at several points of his skin (cutis).

14. The apparatus (100) according to any of the claims 6 to 12, comprising
(a) a first half (110) arranged vertically and having a protection surface (112) for the user transparent for the radiation, and UV radiation sources (114) emitting radiation;
(b) a second half (120) arranged vertically and having a transparent protection surface (122) for the user transparent for the radiation, and UV radiation sources (124) emitting radiation;
(c) whereby the second half (120) may be opened with respect to the first half (110) for allowing a user to get in and may be closed before initiating the irradiation process, or both halves may be moved apart from each other for allowing a user to get in and may be closed by moving them towards each other before initiating the irradiation process;
(d) control apparatus (130) for controlling the time of the irradiation process, which may be operated by the user; and
(e) one or more means (108) for separately controlling the power of the UV radiation sources (114, 124), wherein a control of the power is effected in accordance with the results of the measurements of the skin color and/or the skin tan status of the user at several points of his skin (cutis).

15. A process for irradiating, to a user's body, radiation comprising a UV radiation portion from UV radiation sources emitting such radiation, said process comprising the step of measuring the status, as far as relevant for determining the UV irradiation dose, of the user's skin at several points of the cutis, and controlling the power of single groups of said UV radiation sources, which can direct their radiation substantially separately onto different parts of the user's body, in accordance with the results of said measurements.

16. The process according to claim 15, comprising the steps of measuring the user's skin color or skin tan status at at least two points of the cutis and controlling the power of two or more than two groups of UV radiation sources in accordance with the results of said measuring steps.

17. The process according to claim 15 or claim 16, comprising the steps of measuring the user's skin color or skin tan status at at least two points of the cutis which are selected from skin of the face, skin of the chest, skin of the back, skin of the belly, skin of the bottom, skin of the arms and skin of the legs.

18. The process according to any of the claims 15 to 17, comprising the steps of measuring the user's skin color or skin tan status by means of one or more means (100) measuring the absorption and/or reflection of radiation irradiated onto the skin and having a defined spectral composition in relation to the irradiated radiation and addressing, in accordance with the results of such measurements, means (108) controlling the power of the UV radiation sources (104).

19. The process according to any of the claims 15 to 18, wherein the skin color and/or the skin tan status of the user are measured before initiating the irradiation.

20. The process according to claim 19, wherein a measurement of the skin type and/or of the skin tan status is effected additionally at least once in the course of the irradiation, preferably intermittently or continuously in the course of the irradiation.

21. The process according to any of the claims 15 to 20 for cosmetic skin irradiation.

22. The process according to any of the claims 15 to 20 for medical skin irradiation.

23. A use of the device (200) according to any of the claims 1 to 5 for measuring the status, as far as relevant for determining the UV irradiation dose, of a person's skin in connection with the irradiation, onto the skin, of radiation comprising a UV radiation portion in a tanning apparatus having artificial UV radiation sources.

24. A use of the device (200) according to any of the claims 1 to 5 for measuring the status, as far as relevant for determining the UV irradiation dose, of a person's skin in connection with the irradiation, onto the skin, of radiation comprising a UV radiation portion in nature.

25. A use of the device (200) according to any of the claims 1 to 5 for measuring the status, as far as relevant for determining the UV irradiation dose, of a person's skin in connection with the medical irradiation, onto the skin, of radiation comprising a UV radiation portion.

26. A use of the device (200) according to any of the claims 1 to 5 for measuring the status, as far as relevant for determining the UV irradiation dose, of a person's skin in connection with the cosmetic irradiation, onto the skin, of radiation comprising a UV radiation portion.

27. The use according to any of the claims 23 to 26 for measuring the color of the skin or the skin tan status of a person's skin.
